(19) United States
(12) Patent Application Publication
(10) Pub. No.: US 2015/0328036 A1
(43) Pub. Date: Nov. 19, 2015

(54) GIRDLE

(71) Applicant: INDACO S.R.L., Filiano (IT)

(72) Inventors: Massimo FRANGI, Suvereto (IT); Roberto FRANGI, Buguggiate (IT)

(21) Appl. No.: 14/410,570

(22) PCT Filed: Apr. 24, 2013
(86) PCT No.: PCT/IB13/53227

§ 371 Date: Dec. 22, 2014

(30) Foreign Application Priority Data
Jul. 3, 2012 (IT) MI2012A001169

(51) Int. Cl. A61F 5/02; A41D 13/05

(52) U.S. Cl. CPC A61F 5/028; A41D 13/0531; A41D 13/0525

(57) ABSTRACT

A girdle comprises a first portion (2) and a second portion (3), a first plurality of strips (4) that extend from the first portion (2) toward the second portion (3) and a second plurality of strips (5) that extend from the second portion (3) toward the first portion (2); the strips of each plurality gathering into at least first (6) and second (7) groups, each being joined to respective first (8) and second (9) joining members; tensioning members (12, 13) fastened to respective girdle portions (2, 3), and adapted to be removably fastened to the other portion (2) and acting upon the joining members (8, 9). Pretensioning members (15, 16) are designed to connect the first (8) and second joining members (9) to the respective tensioning members (12, 13), at least the pretensioning members (15, 16) that connect the first (8) or the second (9) joining members to the respective tensioning members (12, 13) being able to be removably fastened to the respective first (2) and second portion (3).

### TECHNICAL FIELD

The present invention relates to a girdle for use in orthopedics for injury rehabilitation and prevention or in other fields (i.e. in sports) for prevention of lower spine and lumbar and abdominal injuries of a user.

### BACKGROUND OF THE INVENTION

Girdles have been known for years and are composed of a belt, usually elastic, which is designed to encircle the waist of a user to assist the spine and the lumbar and abdominal muscles in torso support.

Especially in therapeutic applications, girdles are required to encircle and adequately support the waist of the user. For this reason, a plurality of sizes (which may be as many as ten) shall be provided for each type of girdle, for the user to select the one that best fits his/her build. Of course, if the user's build changes (i.e. due to weight loss or increase) during use of the girdle, a girdle of a different size might have be purchased. Furthermore, girdle manufacturers shall be able to ensure availability of all sizes, which increases manufacturing and logistics costs.

Another important characteristic of girdles is the possibility of making adjustments such that the girdle perfectly fits the anatomy of the user. Indeed, even people of the same size have their own particular anatomy (e.g. more or less prominent hips, lower or higher waist lines, and so on). The possibility of making adjustments should be further combined with the possibility of easily wearing the girdle. In this respect, WO2006119827 discloses a corset comprising two substantially identical portions, each having a plurality of laces extending therefrom at the back portion (i.e. the one designed to be located near the spine of the user) of the corset. The laces that extend from the first portion of the corset cross with those that extend from the second portion (and vice versa) and are gathered into two groups, each connected to a loop engaged by a respective belt for tensioning the second portion of the corset. Each pair of belts for tensioning a portion of the corset (an upper portion and a lower portion) can be also released from the other portion of the corset, when the latter encircles the waist of user. Thus, the two corset portions are joined together, and as the tensioning belts are tightened or loosened when the corset is being closed, corset compression may be increased or released. Also, by setting different tensions for the upper and lower tensioning belts, different compression degrees and lengths may be obtained for the lower and upper portions of the corset, whereby the latter can be conformed to different anatomies.

While this prior art corset solves most of the above mentioned problems, it still suffers from certain drawbacks.

In order to obtain different compression degrees and lengths for the upper and lower portions of the corset while maintaining an adequate tensile and structural symmetry in the corset, the tension of tensioning belts should be accurately calibrated when the corset is being closed, which would require repeated tension adjustments for the upper and lower belts. This cannot be always done easily, whereby the corset does not always perfectly fit the anatomy of the user.

Furthermore, the force that the user is required to apply on the tensioning belts to close the corset is rather important, though not excessive. Therefore, older or physically impaired people might not be independent in wearing the corset. In the light of the above prior art, the technical purpose of the present invention is to provide a girdle that obviates the above mentioned drawbacks.

Particularly, the object of the present invention is to provide a girdle that affords easy differentiated adjustment of the upper and lower portions of the girdle.

A further object of the present invention is to provide a girdle that can be easily used by elder and physically impaired users.

Another object of the present invention is to provide a girdle that can cover a wide range of sizes.

Yet another object of the present invention is to provide a girdle that can fit the peculiar anatomy of the user.

### SUMMARY OF THE INVENTION

According to the present invention, the technical purpose and the intended objects are fulfilled by a corset having the features of one or more of the annexed claims.

### BRIEF DESCRIPTION OF THE DRAWINGS

The features and advantages of the invention will appear from the following detailed description of one embodiment, which is illustrated without limitation in the annexed drawings, in which:

FIG. 1 shows a plan view of a girdle of the present invention, in a first operating configuration,

FIG. 2 shows the girdle of FIG. 1 in a second operating configuration;

FIG. 3 is a perspective view of the girdle of FIG. 1, with certain parts omitted to better show other parts; and

FIGS. 4, 5 and 6 are schematic views of the steps for wearing the girdle of FIG. 1.

### DETAILED DESCRIPTION OF THE INVENTION

A girdle according to the present invention has been generally designated by numeral 1.

The girdle 1 comprises a first portion 2 and a second portion 3, each in band form. The two portions 2, 3 are made of a flexible, non-elastic material, for them to conform to the curves of a user, without elastically extending to an appreciable or considerable extent. The two portions 2, 3 have free ends 2a, 3a, that are able to be removably fastened together. As used herein, the term removably fastened is intended to indicate a connection between two elements that may be made and effected by a user and may be removed by the user him/herself. In the preferred embodiment of the invention, such type of fastening arrangement is given by a hook fabric area disposed on one of the two elements to be fastened, and designed for connection to a corresponding area of the other element that is made of a hairy fabric (or vice versa), to form a Velcro® connection.

A first plurality of strips 4 and a second plurality of strips 5 extend from respective ends 2b, 3b of the first and second portions 2, 3 opposite to the above mentioned free ends 2a, 3b. The first plurality of strips 4 extends toward the second portion 3 and the second plurality of strips 5 extends toward the first portion 2. These strips are also made from a flexible, non-elastic material. The ends of the strips of the first 4 and second 5 pluralities gather into at least first 6 and second 7 groups respectively, each being joined to respective first 8 and second 9 joining members.

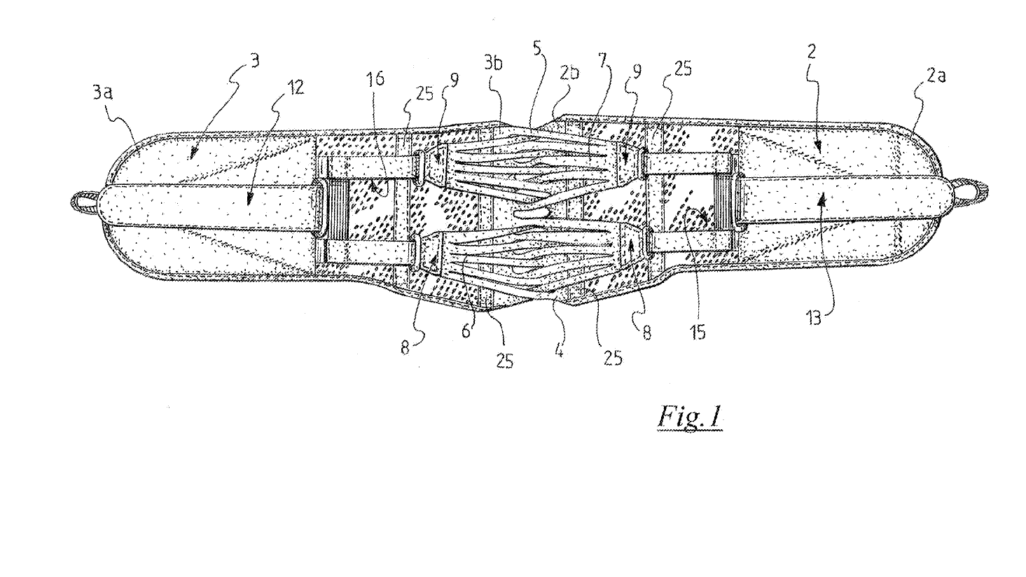

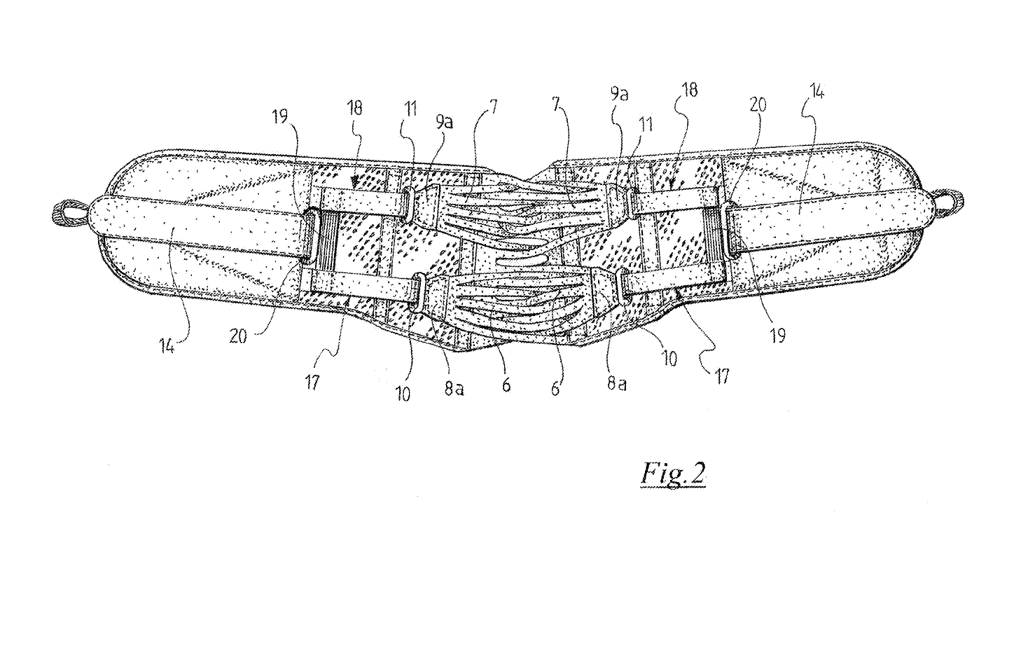

In the preferred embodiment of the invention, two first joining members 8 and two second 9 joining members are provided. The first joining members 8 are placed at the lower portion of the girdle (the one that is proximal to the pelvis of the user) and the second joining members 9 are placed at the upper portion of the girdle (the one that is distal from the pelvis of the user), as shown in FIG. 1. Therefore, a first 8 and a second 9 joining members are both subtended by the first portion 2 of the girdle, as well as by the second portion 3 of the girdle. It shall be noted that the strips of the first plurality of strips 4 alternate (by crossing) with the strips of the second plurality of strips 5, such that the strips of the first plurality cannot disengage from those of the second plurality and vice versa, thereby mutually fastening the two portions 2, 3 of the girdle. The joining members 8, 9 include a retaining portion 8a, 9a (see FIG. 2) in which the ends of the respective groups of strips are firmly fastened together. The joining members 8, 9 further include respective loops 10, 11, which are designed to slidingly engage a respective strap 17, 18, a better described hereinafter. These loops 10, 11 are firmly fastened to the retaining portions 8a, 9a and extend away therefrom.

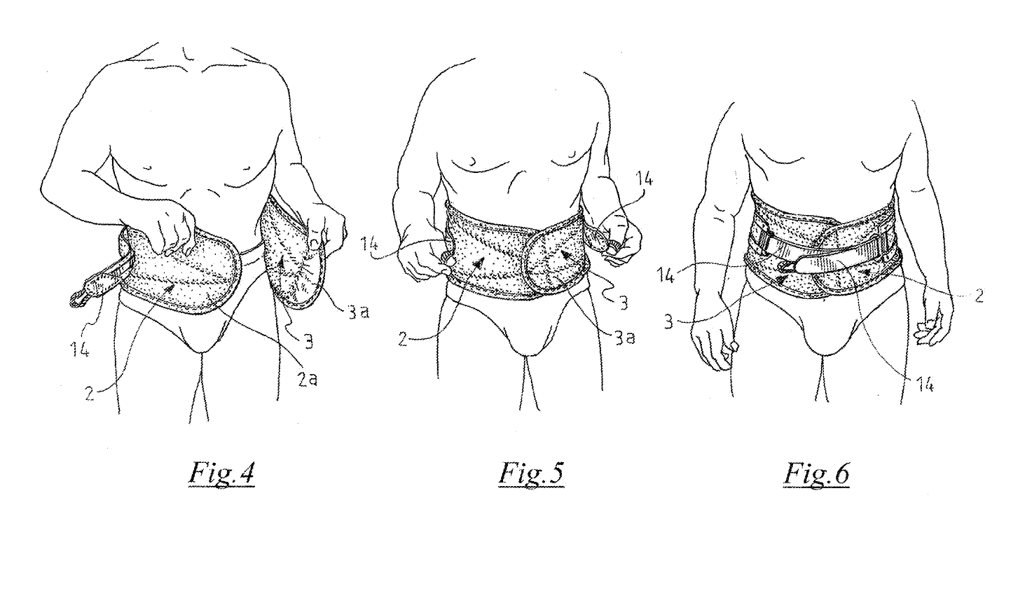

The girdle 1 further comprises tensioning members 12, 13, each being fastened to one of the two portions 2, 3 of the girdle. Each tensioning member 12, 13 is also adapted to be removably fastened (as described above) to the portion 2, 3 of the girdle to which it is not permanently fastened, and operates on the first 8 and second 9 joining members that are subtended by the portion 2, 3 to which it is permanently fastened. Namely, a first tensioning member 12, 13 is permanently fastened to the first portion 2 of the girdle, and adapted to be removably fastened to the second portion 3, and operates on the first 8 and second 9 joining members that are subtended by the first portion 2 of the girdle. Likewise, a second tensioning member 13, 13 is permanently fastened to the second portion 3 of the girdle, and adapted to be removably fastened to the first portion 3, and operates on the first 8 and second 9 joining members that are subtended by the second portion 3 of the girdle. The tensioning members are also made from a flexible, non-elastic material. In the preferred embodiment of the invention, there are two tensioning members. Particularly, each tensioning member comprises a strap 14 (see FIGS. 2 and 4) having a first end 14a fastened to its respective girdle portion and a second end 14b designed to be held by a user. The second end 14b is adapted to be removably fastened to a corresponding area of a girdle portion or the other strap. By actuating the straps 14, the user may encircle the girdle around his/her waist, as schematically shown in FIGS. 5 and 6.

Advantageously, the girdle comprises pretensioning members 15, 16 for connecting the first and second joining members 8, 9 to their respective tensioning members 12, 13. At least the pretensioning members 15, 16 that connect the first 8 or the second joining members 9 to their tensioning members 12, 13 can be removably fastened to the first 2 and second 3 portions respectively in band form. The pretensioning members 15, 16 that are not designed to be removably fastened to the first 2 an second 3 portions respectively of the girdle are stably and irremovably fastened to the first 2 and second 3 portions respectively of the girdle. Thus, as more clearly shown hereinafter, the girdle can fit the particular anatomy (hip width relative to the waist) of the user. In the preferred embodiment of the invention, as shown in the figures, all the pretensioning members 15, 16 are adapted to be removably fastened to the first 2 and second 3 portions of the girdle respectively. Thus, the user may fasten the pretensioning members 15, 16 in different positions on the portions 2, 3 of the girdle, thereby providing different tensioning degrees on the plurality of strips 4, 5, reflecting into different relative positions of the first 2 and second 3 positions of the girdle, such that the girdle may be configured before being worn. Indeed, as mentioned above, the pretensioning members 15, 16 interact with the joining members 8, 9 which are stably fastened to the groups 6, 7 of pluralities of strips 4, 5, thereby interacting with the pluralities of strips 4, 5. Furthermore, as mentioned above, as the girdle is being closed the joining members 8, 9 are actuated by a respective tensioning member 12, 13. Since the pretensioning members 15, 16 also interact with the latter, the above described preconfiguration of the girdle is not altered as the girdle is being worn. In other words, the pretensioning members 15, 16 are configured as transfer members between the tensioning members 12, 13 and the joining members 8, 9, thereby allowing preconfiguration of the relative positions of the two portions 2, 3 of the girdle by adjusting the relative distance between each joining member 8, 9 and its respective tensioning member 12, 14 (as more clearly shown hereinafter). The pretensioning members are also made from a flexible, non-elastic material.

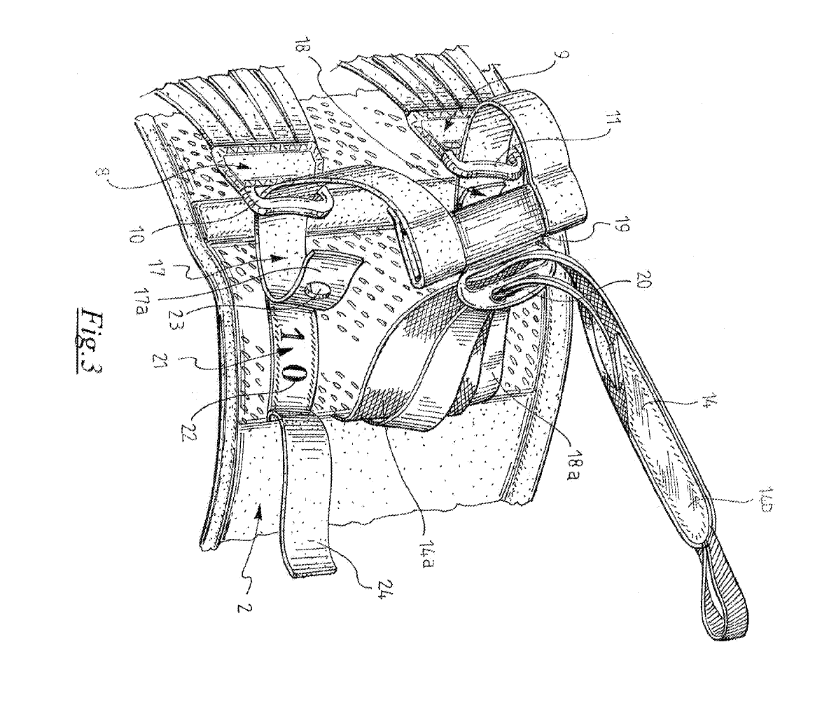

In the preferred embodiment of the invention, each pretensioning member comprises first 17 and second 18 straps, each having a free end 17a, 18a (see FIGS. 2 and 3). The free ends 17a, 18a of the straps 17, 18 are adapted to be removably fastened to the first or second portion of the girdle respectively, as shown in FIG. 3, which shows the free end 17a of the first strap 17 as it is fastened. Opposite to its free end, each strap 17, 18 is fastened to a joining element 19 that integrally joins the two straps 17, 18. A loop 20 is applied to the joining element 19, and slidingly receives the strap 14 that defines a respective tensioning member. In an intermediate position between the free end 17a, 18a and the joining element 19, each strap is slidingly inserted in the loop 10, 11 of the joining elements 8, 9.

It shall be noted that the two straps 17, 18 and the joining element 19 allow the strap 14 to act upon two groups of pluralities of strips 4, 5. Thus, the user may act on one element only, i.e. a strap 14, to stretch both groups of strips 6, 7, which will considerably facilitate the girdle wearing process. Furthermore, as mentioned above, the straps 17, 18 allow preconfiguration of the mutual orientation of the two portions 2, 3 of the girdle. This will also allow both the two portions 2, 3 to be moved toward or away from each other (for preconfiguration of the girdle in various sizes), and the upper portion of the girdle to be tightened or loosened relative to the lower portion (for fitting the girdle to the particular anatomy of the user). Furthermore, these steps are advantageously carried out before wearing the girdle, and hence may be performed in a very easy and convenient manner.

For easier fastening of the straps 17, 18, indexing members 21 are provided (see FIG. 3). These indexing members 21 include a graded scale 22 (given by progressive numbers in FIG. 3), representative of the degree of pretensioning of the pluralities of strips. For accurate positioning of the strap on the selected index, each strap has a hole 23 (see FIG. 3), which allows viewing of the index of the graded scale 22 on which the strap has been fastened. Furthermore, in order to prevent the strap from being released from the selected position due to accidental shocks, an appendage 24 is designed to be removably fastened to the upper surface (i.e. the surface opposite to the one that engages the graded scale) of the strap. This appendage 24 is firmly fastened at one end to the corresponding portion 2, 3 of the girdle. When the straps are placed at the same indices of the grades scale, the first groups of strips 6 and the second groups of strips 8 are pretensioned to the same extent. This will allow the girdle to fit to size, i.e. to be adjusted according to the user's size. When the straps 17, 18 engage different indices of the graded scale, the upper portion of the girdle is loosened or tightened relative to the lower portion, whereby the girdle is adjusted according to the anatomy of the user (more or less narrow hips, more or less pronounced belly).

For perfect support of the user body in the region in contact with the girdle, the latter comprises a plurality of stiffening shafts 25 in pockets formed on the first 2 and second 3 portions of the girdle. Advantageously, all the stiffening shafts 25 are located between their respective girdle portion and the pretensioning members 15, 16 and/or the pluralities of strips 4, 5. Thus, when the girdle is worn, the stiffening shafts 25 are pressed in position by the pretensioning members 15, 16 themselves and/or by the pluralities of strips 4, 5.

In operation, the pretensioning steps as described above and schematically shown in FIG. 3 are carried out first. Thus, the user seizes the two portions 2, 3 of the girdle and draws them near his/her waist thereby partially encircling the latter, as shown in FIG. 4. The user overlaps the free ends 2a, 3a of the girdle portions and actuates the removable fastening arrangement (of the Velcro® type, as described above). This will cause the girdle to encircle the waist of the user in a closed loop and to remain in position (see FIG. 5). Then the user seizes the straps 14 and pulls them toward the overlapped ends of the girdle portions (see FIG. 5). In this step, the straps act upon the pretensioning member/s 15, 16 (that were adjusted beforehand), and the latter will act upon the joining members. 8, 9, that will stretch the pluralities of strips 4, 5. The pluralities of. strips 4, 5 are stretched as controlled by the pretensioning member (i.e. to the same extent like in the configuration of FIG. 1 or to different extents for the upper and lower portions of the girdle, like in the configuration of FIG. 2). If the straps 14 are still pulled, the two girdle portions 2, 3 will be drawn further toward each other and thus allow the girdle to tightly fit on the user's body. Finally, the ends of the straps 14 are removably fastened to each other to maintain the tension so achieved (see FIG. 6). Those skilled in the art will obviously appreciate that a number of changes and variants may be made to the above described configurations, to meet incidental and specific needs. For example, three or more groups of pluralities of strips may be provided for each portion of the girdle, with as many joining members corresponding thereto. In this case, the pretensioning members will include a corresponding number of straps, which are fastened together by a single joining element. Also, if four or more groups of strips are provided for each girdle portion, two pretensioning members may be provided for each girdle portion, which are acted upon by two corresponding straps. All of these variants and changes shall be contemplated in the scope of the invention, as defined in the following claims.

1. A girdle comprising:
a first portion and a second portion each of which is in band form; a first plurality of strips that extend from the first portion towards the second portion and a second plurality of strips that extend from the second portion towards the first portion;
ends of the strips of each plurality of strips being collected into at least first and second groups each of which is joined to respective first and second joining members; tensioning members fixedly connected to the respective first or second portion in band form, able to be removably fastened to the second or first portion and active on said first and second joining members to allow a user to close said first and second portion in band form in a loop;
wherein pretensioning members are designed to connect said first or second joining members to the respective tensioning members, at least the pretensioning members that connect the first or the second joining members to the respective tensioning members being able to be removably fastened to the respective first and second portion in band form.

2. The girdle of claim 1, wherein the pretensioning members that connect both the first and the second joining members to the respective tensioning members can be removably fastened to the respective first and second portion in band form.

3. The girdle of claim 2, comprising indexing members to fixedly connect said pretensioning members to the respective first and second portion in band form in predefined positions.

4. The girdle of claim 3, wherein each pretensioning member comprises a first portion that slidably engages a respective tensioning member, at least two intermediate portions that respectively slidably engage the first and the second joining member and at least two end portions able to be removably fastened to the first or second portion in band form.

5. The girdle of claim 4, wherein each pretensioning member comprises a first and a second strap, each of which is equipped with a free end defining one of said two end portions; said first and a second strap being fixedly connected, at the opposite side with respect to the free ends, to a joining element defining said first portion; a loop being fixedly connected to said joining element to slidably receive the tensioning member.

6. The girdle of claim 5, wherein said first and second joining members comprise respective loops engaged by a respective strap of said pretensioning members; said loops engaging said straps in intermediate positions between the free ends and the joining element.

7. The girdle of claim 6, comprising a plurality of stiffening shafts arranged between the first or the second portion in band form and said pretensioning members or said plurality of strips.

8. The girdle of claim, wherein each tensioning member comprises a strap having a first end fixedly connected to the first or second portion in band form and a second end arranged to be actuated by a user; an intermediate portion of said strap engaging a respective pretensioning member.

9. The girdle of claim 6, wherein said plurality of strips, said pretensioning members and said tensioning members are made from flexible and non-elastic material.

10. The girdle of claim 6, wherein said pretensioning members and said tensioning members are arranged, with respect to said portions in band form, on the opposite side with respect to the body of the user when the girdle is in use.
